(19) United States
(12) Patent Application Publication
(10) Pub. No.: US 2021/0267795 A1
(43) Pub. Date: Sep. 2, 2021

(54) APPARATUS AND METHODS FOR ACCESSING AND TREATING BODILY VESSELS AND CAVITIES

(71) Applicant: CrossBay Medical, Inc., San Diego, CA (US)

(72) Inventor: Steven R. BACICH, Half Moon Bay, CA (US)

(73) Assignee: CrossBay Medical, Inc., San Diego, CA (US)

(21) Appl. No.: 17/324,010

(22) Filed: May 18, 2021

Related U.S. Application Data
(63) Continuation of application No. 15/841,163, filed on Dec. 13, 2017.
Application No. 15/841,163 is a continuation of application No. PCT/US2016/037715, filed on Jun. 15, 2016.
(60) Provisional application No. 62/175,534, filed on Jun. 15, 2015.

(51) Int. Cl. A61F 7/12; A61B 18/04; A61F 7/00

(52) U.S. Cl. CPC A61F 7/123; A61B 2018/00023; A61F 7/007; A61B 18/04

(57) ABSTRACT

Devices and methods for accessing and treating bodily vessels and cavities are disclosed. The devices can have everting balloon catheters that can deliver heating or cooling to biological vessels.

### CROSS-REFERENCE TO RELATED APPLICATION

This application is a continuation of U.S. patent application Ser. No. 15/841,163, filed Dec. 13, 2017, which is a continuation of International Application No. PCT/US2016/037715, filed Jun. 15, 2016, which claims priority to U.S. Provisional Application No. 62/175,534, filed Jun. 15, 2015, all of which are incorporated by reference herein in their entireties.

### BACKGROUND

### 1. Technical Field

An everting catheter is disclosed that can be used for accessing and treating vessels, as examples, the fallopian tubes for contraception, the uterine cavity for the treatment of excessive menorrhagia, the arterial system for the treatment of plaque, the venous system for the treatment of valve disorders, sinus passageways for the treatment of sinusitis, and additional passageways in the mammalian body including the urethra, ureters, bile ducts, mammary ducts, gastrointestinal tract for the treatment of disorders or tissue therapy.

An everting catheter is disclosed for accessing and treating vessels and cavities in combination with other instruments, media, therapeutic agents, and devices which can be equally delivered or placed for treatment or therapy.

### 2. Related Art

For physicians and medical professionals, accessing systems for vessels and bodily cavities in patients have typically used various guidewire and catheter technologies. In the techniques described above, the methods involved pushing an object, guidewire, mandrel, or device itself through the vessel to gain access to a desired region in the body. The result of pushing an object, mandrel, or device creates shear forces on the lumen wall. In some cases the shear forces can result in trauma, pain for the patient, or perforation. In additions, the tortuosity and attributes of the physical anatomy may make access to the desired therapeutic site difficult and challenging.

In contrast, another access technology is referred to as an everting catheter. Everting catheters utilize a traversing action in which a balloon is inverted and with the influence of hydraulic pressure created by a compressible or incompressible fluid or media inside of the balloon, rolls inside out or everts with a propulsion force through the vessel. Everting balloons have been referred to as rolling or outrolling balloons, evaginating membranes, toposcopic catheters, or linear everting catheters as seen in U.S. Pat. Nos. 5,364,345, 5,372,247, 5,458,573, 5,472,419, 5,630,797, 5,902,286, 5,993,427, 6,039,721, 3,421,509, and 3,911,927, all of which are incorporated by reference herein in their entireties. These will all be categorized as everting balloons and due to their property of traversing vessels, cavities, tubes, or ducts in a frictionless manner.

In other words, an everting balloon can traverse a tube without imparting any shear forces on the wall being traversed. Because of this action and lack of shear forces, resultant trauma can be reduced and the risk of perforation reduced. In addition, as a result of the mechanism of travel through a vessel, material and substances in the proximal portion of the tube or vessel are not pushed or advanced forward to a more distal portion of the tube or vessel. Furthermore, as the everting catheter deploys inside out, uncontaminated or untouched balloon material is placed inside the vessel wall.

In the inverted or undeployed state, the balloon is housed inside the catheter body and does not come into direct contact with the patient or physician. As the balloon is pressurized and everted, the balloon material rolls inside out without contacting any element outside of the vessel. The method of access for an everting balloon can be more comfortable for the patient since the hydraulic forces “pull” the balloon membrane through the vessel or duct as opposed to a standard catheter that needs to be “pushed” into and through the vessel or duct.

Due to its ability to navigate tortuous anatomy and gain access to difficult regions of the body, the everting balloon can be a useful tool for physicians to provide therapeutic tools to these regions. In another respect, the everting balloon can be adapted to become the therapeutic tool or device once in the desired location in the body.

One form of therapy is thermal or ablative treatments. Hyper-therapy, by way of heated thermal energy, causes cellular necrosis or a wound-healing response that can promote a desired therapeutic effect. As an example, heated balloons applied in the uterine cavity for the treatment for menorrhagia. Conversely, hypo-therapy, or the cooling of tissue, can promote cellular necrosis and disruption. As an example, cellular disruption of the venous valves can have a positive aesthetic effect in the treatment of varicose veins.

### SUMMARY OF THE INVENTION

Devices and methods for accessing and treating bodily vessels and cavities are disclosed. The devices can have everting balloon catheters that can deliver heating or cooling to biological vessels.

For both hyper- or hypo-therapy, the therapeutic effect can be administered to the everting balloon once access to the desired location has been established by filling the balloon with either heated or cooled media. Alternatively the second catheter can provide the element for heating or cooling the balloon media. The element can be an electrode or an electrically coupled instrument for direct heating, RF, or microwave energy.

Another mechanism for creating a thermal effect is to make the balloon material itself an electrode by plating the balloon surface with flexible electrodes. As an electrode, the balloon can provide radiofrequency, bipolar or mono polar, capacitive coupling, or microwave energy. As an example, the electrodes can be placed onto or within the balloon material. Alternatively the second catheter can provide the electrode, or microwave antenna as an example, that provides the energy through the balloon material and onto the target tissue once the eversion process, or access, has been achieved. Alternatively the second catheter can deliver the electrode that energizes the balloon material to affect the target tissue.

Another example can utilize the everting balloon to provide laser energy at a specific wave length that can be absorbed specifically by chromophores within the desired tissue. The everting balloon would deliver the laser within the second catheter and once energized, provide light energy at a specific wave length for the desired therapeutic tissue.

The above examples utilize the ability of the everting balloon to reach a target site and supply a therapeutic effect. The following examples provide further details for site specific applications.

### Accessing and Treating the Fallopian Tube

The everting catheter can access the fallopian tube with either a hysteroscope or under ultrasound or radiographic guidance. Once everted into the fallopian tube, the media within the everting balloon can be placed by heated or cooled media for tissue necrosis depending upon the amount of time in contact with the target tissue. Representative samples of internal heating of the fallopian tube include U.S. Publication No. 2010/0217250 and U.S. Publication No. 2013/0123613, both of which are incorporated by reference herein in their entireties. Internal fallopian tube heating can include depositing a tubal occlusion member after internally heating the fallopian tube to induce a tissue response.

Everting catheters can have a handle for controlling instruments within an everting catheter, as shown for example in U.S. Pat. No. 5,346,498 which is incorporated by reference herein in its entirety. The handles and instruments can be used to place electrodes within the everting catheter or controlling both the everting balloon and an electrode instrument.

The everting balloon can be configured to have an outer diameter from about 0.5 mm to about 3 mm for example with an outer diameter of less than 2 mm, such as when used in a fallopian tube. The everting balloon can have a length from about 1 cm to about 15 cm, for example depending upon the desired distance or target location in the target site, for example in fallopian tube whether that is the intramural portion, isthmic, ampullary, or fimbria. An everting balloon that exits the fimbria can be in close proximity to the ovary and into the peritoneal cavity of the patient.

The everting catheter mechanism of traversing a vessel can access the uterine cavity via the cervix. The cervical canal is a single lumen vessel that can stretch or dilate. To cross the cervical canal, the everting catheter can have an outer catheter, an inner catheter, an everting balloon membrane, and a handle advancement and pressurization system.

The device can have an adapter, such as a Tuohy-Borst adapter and/or Y-connector, to connect an inner catheter to the balloon membrane. The adapter can allow the inner catheter to advance and retract, for example, through the Y-connector, without losing pressure. The inner catheter can have an internal lumen or be configured as a flexible solid rod or mandrel. The inner catheter can withstand both hydraulic pressures and advancement and retraction tensile and compression forces without deformation. Movement of an advancement button on a handle can move the inner catheter within the Y-connector and through the outer catheter, for example rolling out the everting balloon to traverse the cervical canal. The advancement button can be attached to an advancing ratchet or a roller wheel geared into or with the inner catheter to allow for incrementally stepped and/or one-way translation of the inner catheter.

The everting balloon membrane can be constructed with varying outer diameters depending upon the application. For applications in the cervical canal, the most proximal portion of the everting balloon outer diameter can have a smaller outer diameter than the remainder of the everting balloon membrane. The everting balloon can be made from an irradiated polyolefin, a thin-wall copolymer such as polyether block amides (e.g., Pebax from Arkema in Colombes, France), or combinations thereof.

For treating the uterus, an everting balloon can be rolled into the uterine cavity in a frictionless manner without shear forces. Once everted, the balloon membrane can be filled with heated or cooled media for tissue necrosis. The balloon membrane or inner catheter can be configured with electrodes for heating, RF, microwave, and other energy sources as described below for the treatment of tissue.

The everting balloon can flare outward. The outer diameter of the proximal length of the everting balloon could be configured with an outer diameter of from about 3 mm to about 6 mm, and the distal-most 2 cm or 3 cm of everting balloon can have an outer diameter from about 10 mm to about 20 mm, for example when used in the urethra to create a seal in the bladder.

The everting balloon can access and cross a stenosis in an arterial or venous blood vessel. Once identified in the proper location, the balloon membrane can be configured to apply energy to the arterial plaque. The balloon membrane can be used to deliver energy to disrupt valves in the venous vessels for the treatment of varicose veins. The device can be delivered to the bile ducts, ureters, urethra, GI tract, sinus passageways, esophagus, mammary ducts, or combinations thereof to deliver energy.

The exterior surface of the everting balloon membrane can have electrode wires, plating, or material within the polymer to transmit electrical energy for heating, for example to treat tissue in contact with or adjacent to the membrane.

The everting balloon membrane can be used to deliver an inner catheter that houses electrodes that are connected to an electrical generator for the transmission of RF, microwave, or direct heating. The inner catheter can deliver a microwave antenna for the transmission of microwave energy. The inner catheter can also house a laser to emit laser energy to targeted tissue.

During and after eversion of the balloon, the hydraulic pressure in the everting balloon can be from about 2 atm to about 5 atm of media pressure. The balloon membrane can have more than 5 atm of media pressure, for example to further distend the bodily cavity, lumen or vessel. This additional distension and space created in vivo can, for example, allow for an expandable electrode within or on the surface of the balloon membrane to expand. The distension forces can create a more uniform shape within the bodily cavity or vessel. By stretching the biological vessel walls under distension, application of thermal therapy can be applied by the balloon membrane throughout the entire surface of the tissue.

Everting balloon catheters can be constructed with an inner catheter with an internal lumen or through-lumen (also spelled “thru-lumen”). The through-lumen can be used for the passage of instruments, media, materials, therapeutic agents, endoscope, guidewires, or other instruments. Everting catheters with through-lumens are known in the art, such as disclosed in U.S. Pat. Nos. 5,374,247 and 5,458,573, both of which are incorporated by reference herein in their entireties.

As an example, the everting balloon catheter can be used to access the fallopian tube or the uterine cavity via the cervix. As the everting balloon unrolls through the cervix, the through-lumen or inner catheter can act as a passage for additional instruments or catheters. Once the everting balloon is pressurized, the inner catheter can be advanced by hand or with a one-handed control system.

As described previously, the movement of the inner catheter can be controlled by use of a handle. The handle can allow the physician to hold the entire catheter system and manipulate the pressurization, movement of components, and de-pressurization of the balloon membrane with one hand. This single-handed control can allow the physician to utilize the other hand for the manipulation of instruments, controlling ultrasound, handling visualization techniques, or depositing materials within the through-lumen by use of another syringe or delivery device mechanism.

As described above, a controller can be attached to the outer catheter. The controller can control the advancement and movement of the everting balloon and inner catheter. Once fully deployed, the inner catheter can be positioned (e.g., housed) at least partially or completely within the controller to allow for easy insertion of other devices into the inner catheter and into the target site (e.g., uterine cavity).

The everting balloon can be used to access the mammary ducts to provide direct heating to target tissue.

The everting balloon can navigate the tortuous anatomy of the GI tract. Once at the desired location, the balloon can be configured to deliver treatment.

In addition, for all of the applications mentioned, it may be useful to combine the therapy with additional therapeutic agents or drugs. The everting balloon can be a conduit for drugs (e.g., for example via the through-lumen and/or from coating on the surface), other therapeutic instruments, endoscopes for internal visualization, and other instruments for biopsies, tissue sampling, aspiration, or combinations thereof.

In one example, the everting balloon can house an endoscope to confirm the target location of the GI tract. The everting balloon can be constructed with a through lumen for providing aspiration for tissue, fluid, or cellular sampling. The everting balloon can then be energized to provide treatment to the target tissue. To ensure a more uniform treatment of the GI wall, the balloon membrane can be pressurized at a level to ensure distension of the vessel. If necessary, the through lumen can be employed to evacuate any tissue sloughing or byproducts of excessive heating during or after the treatment.

A thermal treatment system is disclosed. The system can have a radially outer catheter, a radially inner catheter slidably translatable inside the outer catheter, an everting balloon, a heater, and a first fluid media heated above 55° C. The everting balloon can be attached at a first end to the outer catheter and at a second end to the inner catheter. The first fluid media can be exposed to the heater and inside of the everting balloon. The system can have a pump configured to pressurize the fluid media. The fluid media can be heated above 80° C.

The heater can have or be an electrode. The electrode can be or have a coil. At least part of the heater can be attached to the inner catheter. At least part of the heater can be located inside of the everting balloon.

The heater can be radially expandable. The heater can be configured to radially bow outward when in a radially expanded configuration.

The everting balloon can have an everting balloon membrane. At least part of the heater can be embedded in or otherwise attached to the everting balloon membrane.

The system can have a cooler. The fluid media can be cooled below 10° C., below 5° C., or below 0° C.

A method for thermal treatment of biological tissue is disclosed. The method can include positioning a device in a target site. The device can have an outer catheter, an inner catheter slidably translatable inside of the outer catheter, and an everting balloon. A first end of the everting balloon can be attached to the outer catheter. A second end of the everting balloon can be attached to the inner catheter. The method can include delivering a media under pressure to the everting balloon. The method can include everting the everting balloon at the target site. The method can include heating the media to or above 55° C.

The heating can include heating the media before and/or after the delivering of the media to the everting balloon. The heating can be performed with an electrode inside of the everting balloon. The media can be heated when the media is in the everting balloon.

The method can include dilating the target site. The dilating of the target site can include comprises expanding the everting balloon at the target site and/or expanding an electrode in the balloon when the balloon is at the target site.

The target site can be a fallopian tube.

### BRIEF DESCRIPTION OF THE DRAWINGS

FIG. 1 is a schematic drawing of a variation of the thermal delivery system.

FIGS. 2a and 2b are cross-sectional views of a variation for deploying the device in a target site.

FIGS. 3a and 3b are cross-sectional views of variations of the distal end of the device in an everted configuration.

FIGS. 4a and 4b are cross-sectional views of a variation of the distal end of the device in an everted configuration with the electrode in radially contracted and radially expanded configurations, respectively.

FIGS. 5a and 5b are cross-sectional views of a variation of the distal end of the device in an everted configuration with the electrode in radially contracted and radially expanded configurations, respectively.

FIGS. 6a through 6c are cross-sectional views of variations of the distal end of the device in an everted configuration.

### DETAILED DESCRIPTION

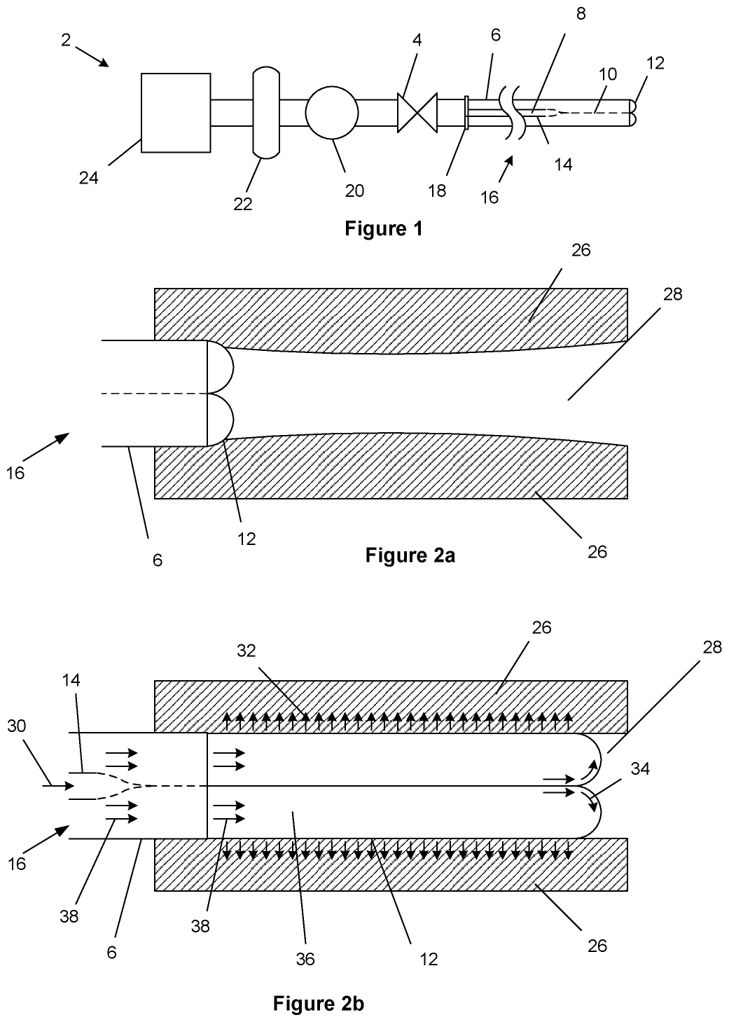

FIG. 1 illustrates that an energy delivery system 2 can have a media reservoir 24 in fluid communication with a heater 22. The heater 22 can be in fluid communication with a pump 20. The pump 20 can be in fluid communication with a media pressure valve 4. The media pressure valve 4 can be in fluid communication with the energy delivery device 16. The heater 22, pump 20, and media pressure valve 4 can be placed in orders other than shown in FIG. 1 (e.g., the media reservoir 24 can connect directly to the pump 20, the pump 20 can then connect downstream directly to the heater 22, and then the heater 22 can connect downstream to the media pressure valve 4). The media reservoir 24, heater 22, pump 20, media pressure valve 4, or combinations thereof can be combined into a single element (e.g., the reservoir can be a pressurized heater 22 with an integrated pump 20 and media pressure valve 4).

The media reservoir 24 can hold media, such as a fluid (e.g., liquid and/or gas), gel, solids (e.g., solid diagnostic agent particles, such as radiopaque and/or echogenic particles), or combinations thereof. The media can be used to pressurize the balloon 10 of the device 16. The media can be saline solution, glycerine, oil, gels, or combinations thereof.

The heater 22 can be a storage tank heater, heat pump heater, or a tankless on-demand heater. The heater 22 can be integrated into the device 16, such as an on-demand heater in the balloon 10 or inner catheter 14 (e.g., an electrode heater). The heater 22 can heat the media from about 55° C. to about to about 100° C., more narrowly from about 50° C. to about 65° C., or from about 80° C. to about 100° C., for example the heater 22 can heat the media, for example, to about 55° C., 80° C. or 100° C. The heater 22 can heat the media above 55° C., 80° C., or 100° C. The system 2 can be thermally insulated so the media temperature in the balloon 10 can be substantially equal to the media temperature in the heater 22. The surface of the balloon membrane 12 can be substantially equal to the media temperature. Tissue being treated can also be changed to substantially the same temperature as the media.

The heater 22 can instead be a cooler, such as a known vapor-compression, evaporative (e.g., swamp) cooler, or thermoelectric refrigeration unit. For example, the heater 22 can be a peltier junction that can heat and/or cool the media. The heater 22 can be gas heater and the system 2 can have a separate vapor-compression refrigeration unit. The cooler can cool the media from about −40° C. to about 20° C., more narrowly from about −10° C. to about 10° C., yet more narrowly from about −5° C. to about 5° C. The cooler can cool the media, for example, to about −40° C., −5° C., 0° C., or 5° C. The cooler can cool the media below −40° C., −5° C., 0° C., 5° C., or 10° C.

The media can be cycled between a heater 22 and a cooler, for example delivering hot media to the balloon 10 for a fixed time and then delivering cool media for a fixed time. The hot and cold cycling can be repeated (e.g., hot media for a fixed time, then cold media for a fixed time, then hot media again for a fixed time, then cold media again for a fixed time). The temperature of the media can be controlled by the operator, for example with a temperature control on a handle of the device 16.

The media pressure valve 4 can be a gate, globe, ball, butterfly, diaphragm, check, needle, or relief valve. The valve can be integrated into the device 16. The valve and/or pump 20 can be controlled to control pressure levels in the balloon 10.

The device 16 can have an outer catheter 6, an inner catheter 14, an everting balloon 10 having a balloon membrane 12, and an adapter 18. The distal end of the inner catheter 14 can be attached to the proximal end of the radially inner portion of the balloon 10. The radially outer portion of the balloon 10 can be attached to the distal end of the outer catheter 6. The inner catheter 14 can be slidably translatable within the outer catheter 6. The proximal end of the outer catheter 6 can be in fluid communication with the remainder of the system 2. The inner lumen 8 or through-lumen of the inner catheter 14 can be accessed through the adapter 18. The adapter 18 can be a Tuohy-Borst adapter 18 and/or Y-connector.

FIG. 2a illustrates that the distal end of the device 16 can be positioned in a biological lumen 28 at the proximal end of a target site having target tissue 26. The target tissue 26 can partially or completely obstruct the biological lumen 28.

FIG. 2b illustrates that the pressurized media 36 can flow, as shown by arrows 38, into the balloon 10, inflating the balloon membrane 12. For example, the media pressure valve 4 can be opened and/or the pump 20 can be turned on to pressurize the media, pushing the media into the balloon 10.

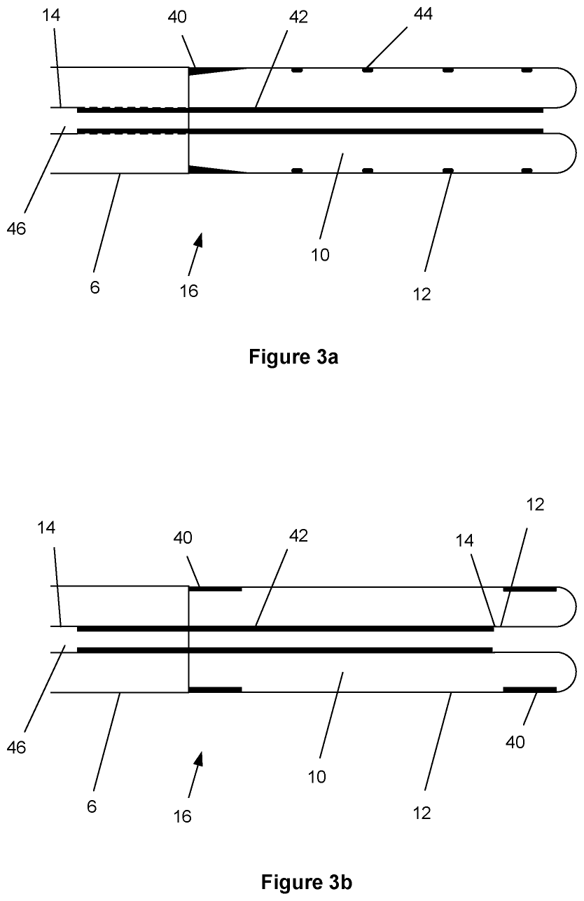

The inner catheter can translate distally, as shown by arrow 30. The inner catheter 14 can be pushed distally and/or the media pressure can cause the balloon 10 to evert and unroll (e.g., hydraulic propulsion), as shown by arrows 34. The unrolling of the balloon 10 can extend the length of the balloon 10 distal to the outer catheter 6. The pressured balloon membrane 12 can expand or distend, as shown by arrows 32, the radially inner surface of the target tissue 26. The balloon membrane 12 can be in contact with or adjacent to the surface of the target tissue 26. The pressure can be sufficient to close a possible radially inner lumen 8 of the balloon 10. As shown in FIG. 3, the radially inner lumen 8 of the lumen can be open and patent, for example, allowing easier passage of agents (e.g., fluid therapeutic or diagnostic agents) or instruments through the through lumen 46 of the inner catheter 14 and balloon 10. Force can be applied to push agents and instruments through the closed lumen of the balloon 10 shown in FIG. 2b and into or distal to the target site.

The heated or cooled media can fill the balloon 10, heating and/or cooling the balloon membrane 12. The balloon membrane 12 can then heat and/or cool the target tissue 26. The media can be heated and/or cooled by the heater 22 and/or cooler in the remainder of the system 2 outside of the device 16 and/or by heating and/or cooling elements in the device 16.

FIG. 3a illustrates that the outer catheter 6 can thermally insulate the media from affecting unintended areas of the patient's body, e.g., not at the target site. The balloon membrane 12 can reduce thermal energy transfer to unintended areas of the patient's body by varying the balloon membrane insulation 40 or thickness in the areas where greater insulation and protection is desired (e.g., at the proximal end of the balloon 10 adjacent to the outer catheter 6). The membrane thickness can be tapered, as shown in FIG. 3a, or discretely stepped, as shown in FIG. 3b. The balloon membrane 12 can be coated with insulating material in the areas of desired thermal protection. The thermal insulation and/or coating can be flexible and expandable enough to evert with the balloon membrane 12. The balloon 10 outer diameter can be made with smaller diameter sections (i.e., waists) to provide less or no tissue contact in certain areas for thermal protection.

One or more thermometers 44, such as thermocouples, can be attached to the external surface of, embedded in, and/or attached to the internal surface of the balloon membrane 12. The thermometers 44 can be used to determine the temperature of the surface of the target tissue 26. The thermometers 44 can be located spread angularly and longitudinally about the balloon membrane 12. For example, the thermometers 44 can be evenly spaced apart longitudinally along a length of the balloon membrane 12 and angularly evenly spaced around the balloon membrane 12,

When the desired location or locations of the target tissue 26 reach the desired temperatures for the desired amounts of time (the physician may wish to make the target tissue 26 a particular temperature merely instantaneously or for an extended time period), the pump 20 and/or valve be reversed and/or the pump 20 can be turned off to reduce the media pressure in the balloon 10. The heater 22 (e.g., heater external to the device 16 and/or electrode 42 or other heater internal to the device 16) and/or cooler can be turned off. The inner catheter can be proximally translated and retracted to frictionlessly invert the balloon 10 into the outer catheter 6. The outer catheter 6 can then be withdrawn from the target site.

The device 16 can have one or more electrodes 42. The electrode 42 can have a cylindrical (as shown) or rod shape. The electrode 42 can be made from an expandable material (e.g., a mesh) that can evert with the balloon membrane 12. The electrode 42 can be attached to the distal end of the inner catheter 14. An electric lead or wire (not shown) extending along the surface of or in the wall of the inner catheter 14 can deliver power to the electrode 42. The electrode 42 can be powered by an electrical power source in the system 2 inside or outside of the device 16.

When the balloon 10 is in an everted configuration, the electrode 42 can be positioned along all or part of the length of the balloon 10. The electrode 42 can extend past the distal end of the balloon 10 or be longitudinally coincidental or, as shown in FIG. 3a, terminate longitudinally proximal to the terminal distal end of the balloon 10.

When the balloon 10 is everted and positioned in contact or adjacent to the target tissue 26, the electrode 42 can be activated by an electrical power source or generator in the system 2. The electrode 42 can then provide RF, microwave, or direct current heating to the media. In RF and microwave applications, for example, the thermal energy can also travel beyond the media and into tissue. Bipolar and monopolar energy can be employed, for example, with the electrode 42 in or attached to the inner catheter 14. RF and microwave energy, for example, can traverse layers of tissue to provide direct heating and thermal treatment deeper than the surface of the target tissue 26. Different wave forms can be used during a single treatment.

The inner catheter 14 can have or be attached to a laser that can deliver laser energy through the balloon membrane 12 to the target tissue 26. The laser can deliver collimated laser light through the inner catheter 14 at various angles for optimal tissue effect. The media can have chromophores. The laser can heat the media, for example by being directed into the media that has chromophores.

FIG. 3b illustrates that the electrode 42 can be in the wall of or attached to the surface of the inner catheter 14, and not longitudinally extend past the distal terminal end of the inner catheter 14. The inner catheter 14 can longitudinally extend past the distal terminal end of the outer catheter 6. The electrode 42 can be located in the through-lumen 46 of the inner catheter 14 and/or balloon 10.

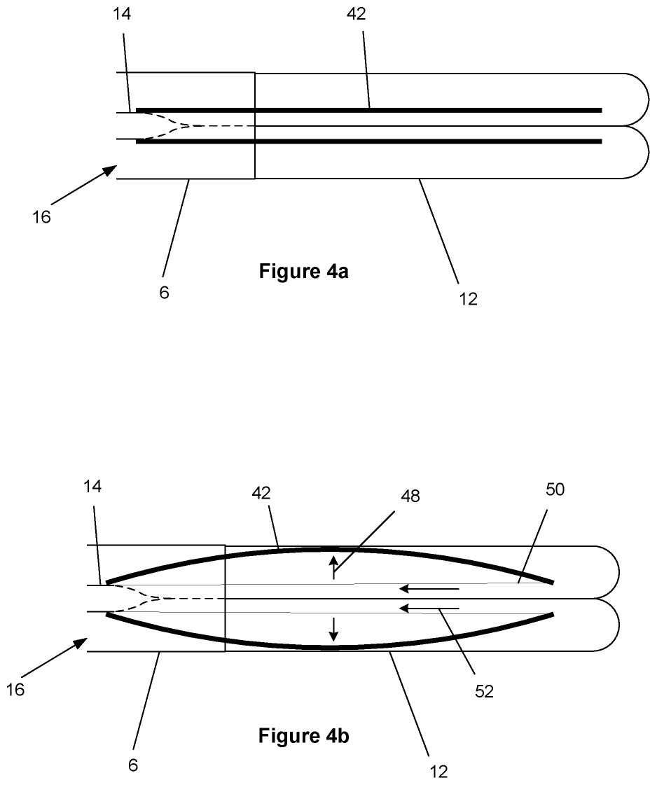

FIG. 4a illustrates that the electrode 42 can be radially expandable and in a radially contracted, unexpanded, unbiased, or relaxed configuration. The electrode 42 can change shape after the balloon 10 is everted.

The electrode 42 can be attached to and extend distally from the distal end of the inner catheter 14. The electrode 42 can extend into the volume of the balloon 10. The balloon 10 can be inflated sufficiently to distend the target tissue 26. One or more pull lines 50 (obscured in FIG. 4a by the electrode 42) or a pull cylinder or tube can be attached to the distal end of the electrode 42 and extend proximally to a control mechanism in or proximal to the inner catheter 14.

FIG. 4b illustrates that the pull lines 50 can be proximally pulled or retracted, as shown by arrows 52. The retraction force exerted on the distal end of the electrode 42 by the pull lines 50 can bow out or radially expand, as shown by arrows 48, the electrode 42. A length or area of the electrode 42 can contact or be adjacent to the radially inner surface of the balloon membrane 12.

Instead or in combination with the pull lines 50, the electrode 42 can be made from a shape memory alloy. When electrical energy is delivered to the electrode 42, the electrode 42 can heat and change to a heated shape. The electrode 42 can also be heated by the body heat of the patient to attain the heated shape. The heated shape can bow out or radially expand as shown in FIG. 4b. When the electrode 42 returns to the original temperature, for example after the electrical energy is no longer delivered to the electrode 42, the electrode 42 can return to the radially unexpanded configuration.

Instead or in combination with the pull lines 50 and shape memory alloy, the electrode 42 can be made from helical coils that unwind for expansion or members that expand when compressed and retract under tension. For example, the electrode 42 can be a helical coil spring. The electrode 42 can be unwound to bow or radially expand. The electrode 42 can be rewound to radially contract.

When the electrode 42 is in the radially expanded configuration, electrical energy can be delivered to the electrode 42 as described above to heat the media and/or the target tissue 26.

The radially expanding electrode 42 can deliver a radially outward force to the balloon membrane 12 and the target tissue 26. For example, the radially expanding electrode 42 can push the balloon membrane 12 radially outward to dilate the biological lumen 28 (e.g., an obstructed blood vessel) in which the device 16 is located.

The electrode 42 can have multiple members. The multiple members can be configured to deliver bipolar RF energy with alternative members being connected to the electrosurgical generator as positive or negative for the delivery of energy.

Once the vessel is dilated, the tissue wall can become stretched or more uniform before delivery of thermal energy. The device 16 can be used to dilate and deliver thermal energy to the walls of the esophagus, GI tract, urethra, other bodily vessels and cavities, and combinations thereof.

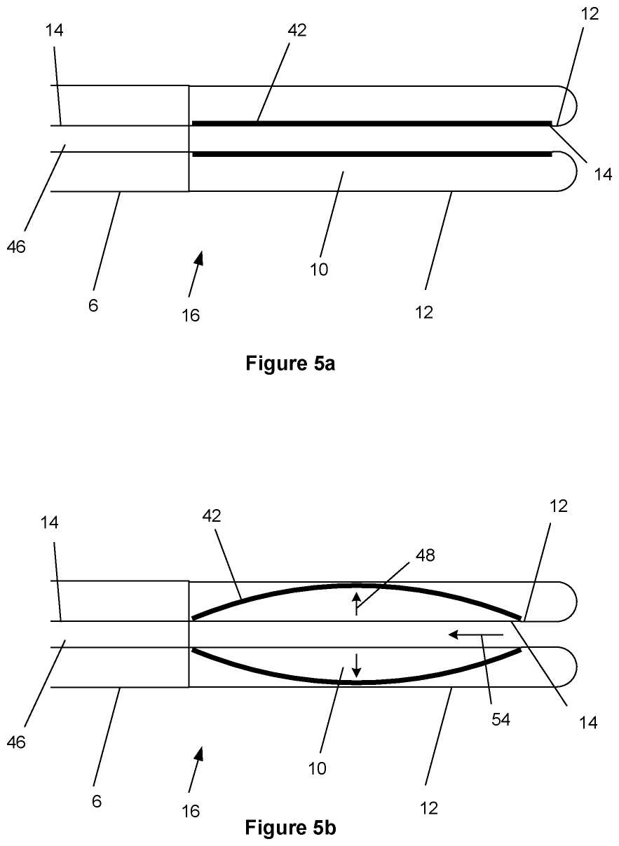

FIG. 5a illustrates that the radially expandable electrode 42 can be attached to the radially outside surface of the inner catheter 14. The distal terminal end of the radially expandable electrode 42 can be equal or proximal to the longitudinal location of the distal terminal end of the inner catheter 14.

FIG. 5b illustrates that the inner catheter 14 can have two parts longitudinally slidable with respect to each other. A first part of the inner catheter 14 can be attached to the distal end of the electrode 42. A second part of the inner catheter 14 can be attached to the proximal end of the electrode 42. The first part of the inner catheter 14 can be longitudinally retracted, as shown by arrow 54, while the second part of the inner catheter 14 is held in a longitudinally constant position with respect to the remainder of the device 16. The retraction of the distal part of the inner catheter 14 radially expandable electrode 42 can bow out or radially expand, as shown by arrows 48, the electrode 42. The retraction of the inner catheter 14 can be used to radially expand the catheter in combination with any of the other methods described herein.

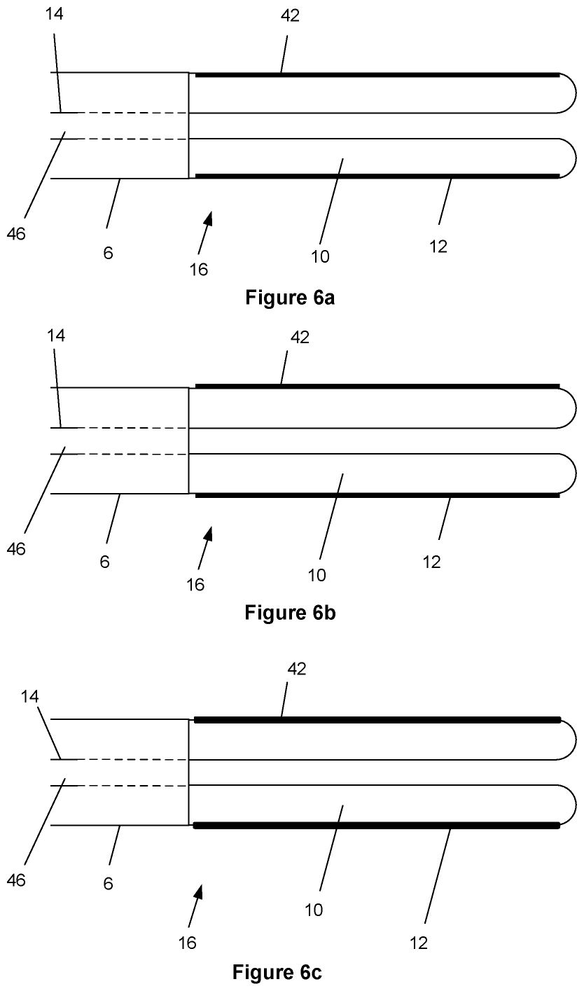

FIG. 6a illustrates that the electrode 42 can be attached to the radially inner surface of the everted balloon membrane 12. FIG. 6b illustrates that the electrode 42 can be attached to the radially outer surface of the everted balloon membrane 12. FIG. 6c illustrates the electrode 42 is embedded in the balloon membrane 12.

The balloon membrane 12 can have an integrated electrically conductive material. For example, a conductive (e.g., metal) plating or wire can be attached to the surface to the balloon membrane 12. Also for example, the balloon membrane 12 can be painted or coated with a conductive material, and/or internally embedded wiring or plating can be embedded into the balloon membrane 12 to act as the electrode 42.

The electrode 42 can be connected to a generator to provide direct heating, RF, or microwave energy. The electrode 42 can be configured to deliver energy in only certain areas or sections of the balloon membrane 12, for example at controllable longitudinal locations along the balloon 10 and angular locations around the balloon 10.

All or part of the everting balloon 10 itself can become the electrode 42, for example if the balloon membrane 12 contains appropriate conductive media. The electrode 42, for example, when the balloon membrane 12 acts as the electrode 42, can deliver uniform heat throughout the entire radially internal surface of the target tissue 26 of a vessel or cavity, for example with varying morphology or curvature. This can provide an electrode 42 that fits the available space and is formed in place inside the vessel or cavity with the media pressure.

The pressure of the media can be increased during use to further inflate the balloon 10, increasing the balloon 10 outer diameter. The distension pressure (i.e., media pressure) can be increased to increase the rigidity of the balloon 10 and decrease the flexibility of the balloon 10. The distension pressure can be reduced to decrease the rigidity and increase the flexibility of the balloon 10. For example, before repositioning the balloon 10 in the target site, the media pressure can be reduced. After repositioning the balloon 10, the media pressure can be increased.

The balloon 10 electrode 42 can be configured as a monopolar electrode 42 with a return pad attached to the patient. As a bipolar electrode 42, the return electrode 42 can be placed on the outer catheter 6 near the distal end of the outer catheter 6.

The term thermal energy and thermal treatment are used herein to refer to the application of heat and/or cold.

It is apparent to one skilled in the art that various changes and modifications can be made to this disclosure, and equivalents employed, without departing from the spirit and scope of the invention. Elements of systems, devices and methods shown with any embodiment are exemplary for the specific embodiment and can be used in combination or otherwise on other embodiments within this disclosure. Furthermore, unless specified otherwise, the elements of methods described can be performed in various orders, not just the disclosed order.

I claim:

1. A thermal treatment system comprising:
a radially outer catheter;
a radially inner catheter slidably translatable inside the outer catheter;
an everting balloon attached at a first end to the outer catheter and at a second end to the inner catheter;
a thermometer attached to and/or embedded in the everting balloon;
a heater;
a first fluid media heated above 55° C., wherein the first fluid media is exposed to the heater and inside of the everting balloon; and
a pump in fluid communication with the heater and configured to pressurize the first fluid media.

2. The system of claim 1, wherein the first fluid media is heated above 80° C.

3. The system of claim 1, wherein the heater comprises an electrode.

4. The system of claim 3, wherein the electrode comprises a coil.

5. The system of claim 1, wherein at least part of the heater is attached to the inner catheter.

6. The system of claim 1, wherein at least part of the heater is located inside of the everting balloon.

7. The system of claim 1, wherein the heater is radially expandable.

8. The system of claim 7, wherein the heater is configured to radially bow outward when in a radially expanded configuration.

9. The system of claim 1, wherein the everting balloon has an everting balloon membrane, and wherein at least part of the heater is embedded in the everting balloon membrane.

10. A method for thermal treatment of biological tissue comprising:
positioning a device in a target site, wherein the device comprises an outer catheter, an inner catheter slidably translatable inside of the outer catheter, and an everting balloon, wherein a first end of the everting balloon is attached to the outer catheter, and wherein a second end of the everting balloon is attached to the inner catheter;
delivering a media under pressure to the everting balloon;
everting the everting balloon at the target site;
heating the media to or above 55° C. with a heater, wherein the media is exposed to the heater and inside of the everting balloon; and
bending the heater, wherein bending the heater comprises compressing the heater.

11. The method of claim 10, wherein the heating comprises heating the media before the delivering the media.

12. The method of claim 10, wherein the heating comprises heating the media after the delivering the media.

13. The method of claim 12, wherein the heating comprises heating the media with an electrode inside of the everting balloon.

14. The method of claim 12, wherein the heating of the media is when the media is in the everting balloon.

15. The method of claim 10, further comprising dilating the target site, and wherein the dilating comprises expanding the everting balloon at the target site, and wherein the dilating comprises expanding an electrode in the balloon when the balloon is at the target site, and wherein the target site comprises a fallopian tube.

16. A thermal treatment system comprising:
a radially outer catheter;
a radially inner catheter slidably translatable inside the outer catheter;
an everting balloon attached at a first end to the outer catheter and at a second end to the inner catheter, wherein a length of the everting balloon is more thermally insulated than the remainder of the everting balloon;
a heater;
a first fluid media heated above 55° C., wherein the first fluid media is exposed to the heater and inside of the everting balloon; and
a pump in fluid communication with the heater and configured to pressurize the first fluid media.
